(19) United States
(12) Patent Application Publication
(10) Pub. No.: US 2023/0381270 A1
(43) Pub. Date: Nov. 30, 2023

(54) PHARMACEUTICAL COMBINATIONS FOR TREATING CANCER

(71) Applicant: POLYPHOR AG, Allschwil (CH)

(72) Inventors: Michael BAUER, Lörrach (DE); Leon HOOFTMAN, Arlesheim (CH); Barbara ROMAGNOLI, Binningen (CH)

(21) Appl. No.: 18/296,104

(22) Filed: Apr. 5, 2023

Related U.S. Application Data
(63) Continuation of application No. 16/486,945, filed on Aug. 19, 2019, now abandoned.

(30) Foreign Application Priority Data
Feb. 20, 2017 (EP) 17020059.6

(51) Int. Cl. A61K 38/12; A61K 9/00; A61K 31/357; A61P 35/00

(52) U.S. Cl. CPC A61K 38/12; A61K 9/0019; A61K 31/357; A61P 35/00

(57) ABSTRACT

The present invention relates to pharmaceutical combinations comprising a compound of formula T or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof and their use in a method for the prevention, delay of progression or treatment of cancer in a subject.

### FIELD OF THE INVENTION

The present invention relates to pharmaceutical combinations comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof and their use in a method for the prevention, delay of progression or treatment of cancer in a subject.

### BACKGROUND OF THE INVENTION

Despite the ever increasing number of cancer therapies in general, and combination cancer therapies in particular, cancer is still the third most common cause of death worldwide after cardiovascular diseases and infectious/parasitic diseases; in absolute numbers, this corresponds to 7.6 million deaths (ca. 13% of all deaths) in any given year. The WHO estimates deaths due to cancer to increase to 13.1 million by 2030, while the American Cancer Society expects over 1,685,210 new cancer cases diagnosed and 595,690 cancer deaths in the United States in 2016.

Metastatic breast cancer (mBC) remains an incurable disease. Despite improvements in early detection, adjuvant therapy, and systemic treatment, more than 40,000 women in the United States alone will die from breast cancer in the next twelve months. Hormonal therapy is the initial course of treatment for those breast cancers that are estrogen receptor positive. Unfortunately, most of these patients become resistant to hormone therapy, and like those patients that are hormone receptor negative, they must then rely upon cytotoxic chemotherapy to control their disease. Despite new targeted therapies and cytotoxic agents that have recently been added to the treatment armamentarium, most patients with metastatic breast cancer develop resistance within months and overall survival remains poor. One of the most recently registered new cytoreductive agents is eribulin (Halaven®). Recently published data regarding the use of eribulin in relapsed mBC patients in 3rd line and beyond, demonstrated that progression-free survival (PFS) and overall survival (OS) could be prolonged 3.7 months and 13.1 months, respectively, and these data formed the basis for registration of this novel chemotherapeutic agent. Despite the recent addition of new therapeutic options, there is a distinct need for new treatment modalities for the treatment of metastatic breast cancer.

### SUMMARY OF THE INVENTION

It has now unexpectedly been found that a combination comprising a compound of formula I or a pharmaceutically acceptable salt thereof such as e.g. eribulin mesylate and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is useful for the prevention, delay of progression or treatment of cancer, in particular breast cancer, metastatic breast cancer, and relapsed metastatic breast cancer. In a phase I escalation study and a subsequent extension study, it was unexpectedly found that treatment with said combination provides a high therapeutic activity with a objective response rate (ORR) of up to 38%.

Taking these findings into account, the present invention is herewith provided in its following aspects.

In a first aspect the present invention provides a pharmaceutical combination comprising:
(a) a compound of formula I

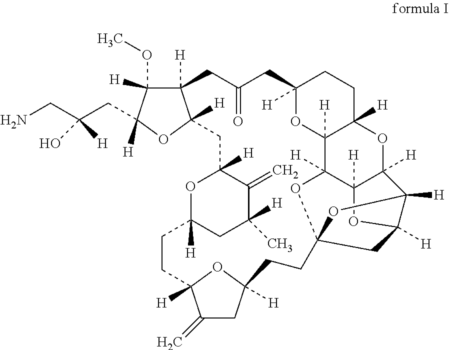

which has the chemical name 2-(3-amino-2-hydroxypropyl)hexacosahydro-3-methoxy-26-methyl-20,27-bis(methylene)11,15-18,21-24,28-triepoxy-7,9-ethano-12,15-methano-9H,15H-furo (3,2-i)furo(2′,3′-5,6)pyrano (4,3-b)(1,4)dioxacyclopentacosin-5-(4H)-one, or a pharmaceutically acceptable salt thereof;
b) cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof; and optionally
(c) one or more pharmaceutically acceptable diluents, excipients or carriers.

In a second aspect the present invention provides a pharmaceutical combination as described herein for use as a medicament.

In a third aspect the present invention provides a pharmaceutical combination as described herein, for use in a method for the prevention, delay of progression or treatment of cancer in a subject.

In a fourth aspect the present invention provides a kit of parts comprising a first container, a second container and a package insert, wherein the first container comprises at least one dose of a medicament comprising a compound of formula I or a pharmaceutically acceptable salt thereof or a compound of formula Ia, the second container comprises at least one dose of a medicament comprising cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cysl 1 or a pharmaceutically acceptable salt thereof and the package insert comprises instructions for treating a subject for cancer using the medicaments, wherein the cancer is selected from the group consisting of breast cancer, metastatic breast cancer, and relapsed metastatic breast cancer.

### DETAILED DESCRIPTION OF THE INVENTION

As outlined above, the present invention provides pharmaceutical combinations comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof which are useful for the prevention, delay of progression or treatment of cancer.

Thus, in a first aspect the present invention provides a pharmaceutical combination comprising:
(a) a compound of formula

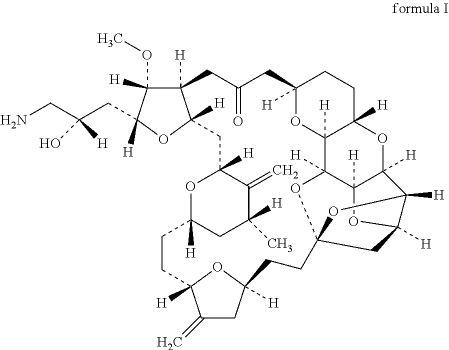

which has the chemical name 2-(3-amino-2-hydroxypropyl)hexacosahydro-3-methoxy-26-methyl-20,27-bis(methylene)11,15-18,21-24,28-triepoxy-7,9-ethano-12,15-methano-9H,15H-furo(3,2-i)furo(2′,3′-5 ,6)pyrano(4,3-b)(1,4)dioxacyclopentacosin-5-(4H)-one, or a pharmaceutically acceptable salt thereof;
(b) cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof; and optionally
(c) one or more pharmaceutically acceptable diluents, excipients or carriers.

For the purposes of interpreting this specification, the following definitions will apply and whenever appropriate, terms used in the singular will also include the plural and vice versa.

It is to be understood that the terminology used herein is for the purpose of describing particular embodiments only and is not intended to be limiting. The terms “comprising”, “having”, and “including” are to be construed as open-ended terms (i.e. meaning “including, but not limited to,”) unless otherwise noted.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The terms “individual,” “subject” or “patient” are used herein interchangeably. In certain embodiments, the subject is a mammal. Mammals include, but are not limited to primates (including human and non-human primates). In a preferred embodiment, the subject is a human.

The term “about” as used herein refers to +/−10% of a given measurement.

The term “dose” as used herein refers to the total amount of an active ingredient (e.g., the compound of formula I or a pharmaceutically acceptable salt thereof or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof to be taken each time by a subject (e.g. a human).

The term “objective response rate” (ORR) as used herein refers to the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

The ORR refers to the sum of complete response (CR) and partial response (PR).

The term “clinical benefit rate” (CBR) as used herein refers to the sum of complete response (CR), partial response (PR) and stable disease (SD)≥6 months.

The term “complete response” (CR) as used herein in relation to target lesions refers to disappearance of all target lesions. Any pathological lymph nodes (whether target or non-target) must have reduction in short axis to <10 mm. The term complete response (CR) as used herein in relation to non-target lesions refers to disappearance of all non-target lesions and normalization of tumor marker level. All lymph nodes must be non-pathological in size (<10 mm short axis).

The term “partial response” (PR) as used herein in relation to target lesions refers to at least a 30% decrease in the sum of the diameters of target lesions, taking as reference the baseline sum diameters.

The term “progressive disease” (PD) as used herein in relation to target lesions refers to at least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progressions. The term progressive disease (PD) as used herein in relation to non-target lesions refers to appearance of one or more new lesions and/or unequivocal progression of existing non-target lesions. Unequivocal progression should not normally trump target lesion status. It must be representative of overall disease status change, not a single lesion increase.

The term “stable disease” (SD) as used herein in relation to target lesions refers to neither sufficient shrinkage to qualify for PR nor sufficient increase to qualify for PD, taking as reference the smallest sum diameters while on study.

The compound of formula I

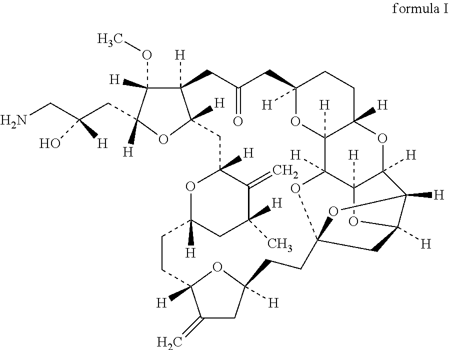

has the chemical name 2-(3-amino-2-hydroxypropyl)hexacosahydro-3-methoxy-26-methyl-bis(methylene)11,15-18,21-24,28-triepoxy-7,9-ethano-12,15-methano-9H,15H-furo(3,2-i)furo(2′,3′-5,6)pyrano(4,3-b)(1,4)dioxacyclopentacosin-5-(4H)-one and is also referred to in the literature as (18,3,5,6S,9S,12S,14R,16R,18S,20R,21R,22S,26R,298,31R,32,5,35R,36,S)-20-[(2S)-3-amino-2-hydroxypropyl]-21-methoxy-14-methyl-8,15-bis (methylene)-2,19,30,34,37,39,40,41-octaoxanonacyclo[24.9.2.1_{3,32}.1^{3,33}.1^{6,9}.1^{12,16}.0^{18,22}.0^{29,36}.0^{31,35}]hentetracontan-24-one; 11,15:18, 21:24, 28-Triepoxy-7,9-ethano-12,15-methano-9H,15H-furo[3,2-i]furo[2′,3′:5,6] pyrano[4,3-b][1,4]dioxacyclopentacosin-5(4H)-one, 2-[(2S)-3-amino-2-hydroxypropyl]hexacosahydro-3-methoxy-26-methyl-20,27-bis(methylene)-, (2R, 3R, 3aS, 7R, 8aS, 9S, 10aR, 11S, 12R, 13aR, 13bS, 15S, 18S, 21S, 24S, 26R, 28R, 29aS); or eribulin (CAS Registry Number: 253128-41-5).

In a preferred embodiment, the compound of formula I is the compound of formula Ia

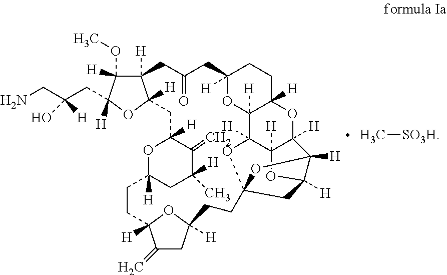

The compound of formula Ia has the chemical name 2-(3-amino-2-hydroxypropyl)hexacosahydro-3-methoxy-26-methyl-20,27-bis(methylene)11,15-18,21-24,28-triepoxy-7,9-ethano-12,15-methano-9H,15H-furo(3,2-i)furo(2′,3′-5,6)pyrano(4,3-b)(1,4)dioxacyclopentacosin-5-(4H)-one methanesulfonate and is also referred to in the literature as (1S,3S,6S,98,12S,14R,16R,18S,20R,21R,22S,26R,29S,31R,32S,35R,36S)-20-[(2S)-3-amino-2-hydroxypropyl]-21-methoxy-14-methyl-8,15-bis(methylene)-2,19,30,34,37,39,40,41-octaoxanonacyclo[24.9.2.1^{3,32}.1^{3,33}.1^{6,9}.1^{12,16}.0^{18,22}.0^{29,36}.0^{31,35}]hentetracontan-24-one methanesulfonate (1:1); 11,15:18, 21:24, 28-Triepoxy-7,9-ethano-12,15-methano-9/1,1 5H-furo[3,2-i]furo[2′,3′:5,6] pyrano[4,3-b][1,4]dioxacyclopentacosin-5(4H)-one, 2-[(2S)-3-amino-2-hydroxypropyl]hexacosahydro-3-methoxy- 26-methyl-20,27-bis(methylene)-, (2R, 3R, 3aS, 7R, 8aS, 9S, 10aR, 11S, 12R, 13aR, 13bS, 155, 185, 21S, 24S, 26R, 28R, 29aS)-, methanesulfonate; or eribulin mesylate (CAS Registry Number: 441045-17-6). Eribulin mesilate is a non-taxane inhibitor of microtubule dynamics of the halichondrin class of antineoplastic drugs. It is a structurally modiEed synthetic analogue of halichondrin B, a natural product isolated from the marine sponge Halichondria okadai. It has a novel mode of action that is distinct from those of other tubulin-targeting agents: inhibiting the microtubule growth phase without affecting the shortening phase, resulting in tubulin sequestration into non-productive aggregates. The compound is approved for the treatment of patients with metastatic breast cancer who have previously received at least two chemotherapeutic regimens for the treatment of metastatic disease. Eribulin mesilate is marketed under the tradename Halaven®.

Cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 is also referred as POL6326 herein or balixafortide. POL6326 is a cyclic synthetic peptide consisting of 16 amino acids and an antagonist of the highly conserved chemokine receptor CXCR4 and is being developed as an IV treatment in combination with chemotherapy in patients with leukemias (autologous transplantation). In vitro receptor binding studies demonstrated a significant affinity of POL6326 for the human CXCR4 receptor, as well as a general lack of significant binding to other potential target receptors.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxy-benzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-enel-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e. g. an alkaline metal ion, an alkaline earth metal ion, or an aluminum ion; or coordinates with an organic base such as ethanolamine, diethanolamine, triethanolamine, tromethamine, N-methylglucamine, and the like.

Particularly suitable pharmaceutically acceptable salts of the compound of formula I are e.g. methane- or ethane-sulfonate. Most preferred is the methanesulfonate salt of the compound of formula I i.e. the compound of formula Ia.

Particularly suitable pharmaceutically acceptable salts of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 to be useful in the context of the present invention include the acetates, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonate, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid. Suitable inorganic acids are, for example, halogen acids, such as hydrochloric acid, sulfuric acid, or phosphoric acid.

The compound of formula I or a pharmaceutically acceptable salt thereof is comprised by the pharmaceutical combination of the present invention. Preferably the pharmaceutical combination of the present invention comprises a pharmaceutically acceptable salt of the compound of formula I. More preferably the pharmaceutical combination of the present invention comprises the compound of formula Ia (eribulin mesylate). Cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or pharmaceutically acceptable salts thereof are comprised by the pharmaceutical combination of the present invention. Preferably the pharmaceutical combination of the present invention comprises cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 in free form. More preferably the pharmaceutical combination of the present invention comprises cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 as acetate salt.

### Combinations

As outlined above, the invention relates to a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof. A pharmaceutical combination according to the invention is for example a combined preparation or a pharmaceutical composition, for simultaneous, separate or sequential use.

The term “combined preparation” as used herein defines especially a “kit of parts” in the sense that the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof can be dosed independently, either in separate form or by use of different fixed combinations with distinguished amounts of the active ingredients. In a preferred embodiment, the pharmaceutical combination according to the invention is a combined preparation.

In a more preferred embodiment the pharmaceutical combination according to the invention is a combined preparation wherein the compound of formula 1 or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof are dosed independently from each other, i.e. are dosed in separate form.

The ratio of the amount of the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof to be administered in the combined preparation can be varied, e.g. in order to cope with the needs of a patient sub-population to be treated or the needs of a single patient, which needs can be different due to age, sex, body weight, etc. of a patient. The individual parts of the combined preparation (kit of parts) can be administered simultaneously or sequentially, i.e. chronologically staggered, e.g. at different time points and with equal or different time intervals for any part of the kit of parts.

The term “pharmaceutical composition” refers to a fixed-dose combination (FDC) that includes the compound of formula I and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, combined in a single dosage form, having a predetermined combination of respective dosages.

The pharmaceutical combination further may be used as add-on therapy. As used herein, “add-on” or “add-on therapy” means an assemblage of reagents for use in therapy, the subject receiving the therapy begins a first treatment regimen of one or more reagents prior to beginning a second treatment regimen of one or more different reagents in addition to the first treatment regimen, so that not all of the reagents used in the therapy are started at the same time. A preferred add-on therapy of the present invention, comprises adding a compound of formula I therapy to a patient already receiving cyclo(-Tyr-His-Ala-Cys-Ser-Ala-^{D}Pro-Dab-Arg-Tyr-Cys-Tyr-Gln-Lys-^{D}Pro-Pro-) therapy.

The amount of the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof to be administered will vary depending upon factors such as the particular compound, disease condition and its severity, according to the particular circumstances surrounding the case, including, e.g., the route of administration, the condition being treated, the target area being treated, and the subject or host being treated.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof wherein said compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof are present in a therapeutically effective amount.

The expression “effective amount” or “therapeutically effective amount” as used herein refers to an amount capable of invoking one or more of the following effects in a subject receiving the combination of the present invention: (i) increase of objective response rate (ORR); (ii) inhibition or arrest of tumor growth, including, reducing the rate of tumor growth or causing complete growth arrest; (iii) reduction in the number of tumor cells; (iv) reduction in tumor size; (v) reduction in tumor number; (vi) inhibition of metastasis (i.e. reduction, slowing down or complete stopping) of tumor cell infiltration into peripheral organs; (vii) enhancement of antitumor immune response, which may, but does not have to, result in the regression or elimination of the tumor; (viii) relief, to some extent, of one or more symptoms associated with cancer; (ix) increase in progression-free survival (PFS) and/or; overall survival (OS) of the subject receiving the combination.

Determination of a therapeutically effective amount is well within the capability of those skilled in the art, especially in light of the detailed disclosure provided herein. In some embodiments, a therapeutically effective amount of the compound of formula I, or a pharmaceutically acceptable salt thereof, may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

The therapeutically effective amount may vary depending on the subject, and disease or condition being treated, the weight and age of the subject, the severity of the disease or condition, and the manner of administering, which can readily be determined by one ordinary skilled in the art.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof wherein said compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof are present in an amount producing an additive therapeutic effect.

As used herein, the term “additive” means that the effect achieved with the pharmaceutical combinations of this invention is approximately the sum of the effects that result from using the anti-cancer agents, namely the compound of formula I and cyclo(-Tyr-His-Ala-Cys-Ser-Ala-D Pro-Dab-Arg-Tyr-Cys-Tyr-Gln-Lys-^{D}Pro-Pro-) having a disulfide bond between Cys4 and Cys11, or a pharmaceutically acceptable salt thereof, as a monotherapy.

Advantageously, an additive effect provides for greater efficacy at the same doses, and may lead to longer duration of response to the therapy.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein said compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof are present in an amount producing a synergistic therapeutic effect.

As used herein, the term “synergistic” means that the effect achieved with the pharmaceutical combinations of this invention is approximately higher than the sum of the effects that result from using the anti-cancer agents, namely the compound of formula I and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, as a monotherapy.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said compound of formula I or a pharmaceutically acceptable salt thereof in the combination is from about 0.1 to about 50 mg or from about 0.1 to about 20 mg or from about 0.1 to about 10 mg or from about 0.5 to about 8 mg or from about 0.5 to about 6 mg or from about 1 to about 2 mg.

In one embodiment, the invention provides a pharmaceutical combination comprising a compound of formula I or a pharmaceutically acceptable salt thereof and cyclo(-Tyr-His-Ala-Cys-Ser-Ala-PPro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said compound of formula I or a pharmaceutically acceptable salt thereof in the combination is about 1 mg, about 1.2 mg, about 1.5 mg, about 2 mg, about 3 mg, about 4 mg, about 5 mg, about 6 mg, about 7 mg, about 8 mg, about 9 mg or about 10 mg, preferably about 1.2 mg.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof in the combination is from about 0.1 to about 50 mg or from about 0.1 to about 20 mg or from about 0.1 to about 10 mg or from about 0.5 to about 8 mg or from about 0.5 to about 6 mg, or from about 1 to about 5.5 mg, or from about 4.5 to about 8 mg, or from about 4.5 to about 5.5 mg, preferably from about 4.5 to about 8 mg or from about 4.5 to about 5.5 mg.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof in the combination is about 1 mg, about 2 mg, about 2.5 mg, about 3 mg, about 3.5 mg, about 4 mg, about 4.5 mg, about 5.5 mg, about 7.5 mg or about 10 mg, preferably about 5.5 mg.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said compound of formula I or a pharmaceutically acceptable salt thereof in the combination is from about 0.1 to about 50 mg or from about 0.1 to about 20 mg or from about 0.1 to about 10 mg or from about 0.5 to about 8 mg or from about 0.5 to about 6 mg or from about 1 to about 2 mg; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof in the combination is from about 0.1 to about 50 mg or from about 0.1 to about 20 mg or from about 0.1 to about 10 mg or from about 0.5 to about 8 mg or from about 0.5 to about 6 mg, or from about 1 to about 5.5 mg.

In a preferred embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said compound of formula I or a pharmaceutically acceptable salt thereof in the combination is about 1 mg, about 1.2 mg, about 1.5 mg, about 2 mg, about 3 mg, about 4 mg, about 5 mg, about 6 mg, about 7 mg, about 8 mg, about 9 mg or about 10 mg; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof in the combination is about 1 mg, about 2 mg, about 2.5 mg, about 3 mg, about 3.5 mg, about 4 mg, about 4.5 mg, about 5.5 mg, about 7.5 mg or about 10 mg. In a particularly preferred embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said compound of formula I or a pharmaceutically acceptable salt thereof in the combination is about 1.2 mg; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof in the combination is from about 4.5 to about 8 mg, from about 4.5 to about 5.5 mg, preferably about 5.5 mg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said compound of formula Ia in the combination is from about 0.1 to about mg or from about 0.1 to about 20 mg or from about 0.1 to about 10 mg or from about to about 8 mg or from about 0.5 to about 6 mg or from about 1 to about 2 mg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said compound of formula Ia in the combination is about 1 mg, about 1.2 mg, about 1.5 mg, about 2 mg, about 3 mg, about 4 mg, about 5 mg, about 6 mg, about 7 mg, about 8 mg, about 9 mg or about 10 mg, preferably about 1.2 mg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said cyclo(-Tyr-His-Ala-Cys-Ser-Ala-^{D}Pro-Dab-Arg-Tyr-Cys-Tyr-Gln-Lys-D Pro-Pro-) having a disulfide bond between Cys4 and Cys11 acetate salt in the combination is from about 0.1 to about 50 mg or from about 0.1 to about 20 mg or from about 0.1 to about 10 mg or from about 0.5 to about 8 mg or from about 0.5 to about 6 mg, or from about 1 to about 5.5 mg, or from about 4.5 to about 8 mg, or from about 4.5 to about 5.5 mg, preferably from about 4.5 to about 8 mg or from about 4.5 to about 5.5 mg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt in the combination is about 1 mg, about 2 mg, about 2.5 mg, about 3 mg, about 3.5 mg, about 4 mg, about 4.5 mg, about 5.5 mg, about 7.5 mg or about 10 mg, preferably about 5.5 mg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said compound of formula Ia in the combination is from about 0.1 to about mg or from about 0.1 to about 20 mg or from about 0.1 to about 10 mg or from about to about 8 mg or from about 0.5 to about 6 mg or from about 1 to about 2 mg; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt in the combination is from about 0.1 to about 50 mg or from about 0.1 to about 20 mg or from about 0.1 to about 10 mg or from about 0.5 to about 8 mg or from about 0.5 to about 6 mg, or from about 1 to about 5.5 mg.

In a preferred embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said compound of formula Ia in the combination is about 1 mg, about 1.2 mg, about 1.5 mg, about 2 mg, about 3 mg, about 4 mg, about 5 mg, about 6 mg, about 7 mg, about 8 mg, about 9 mg or about 10 mg; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt in the combination is about 1 mg, about 2 mg, about 2.5 mg, about 3 mg, about 3.5 mg, about 4 mg, about 4.5 mg, about 5.5 mg, about 7.5 mg or about 10 mg.

In a particularly preferred embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said compound of formula Ia in the combination is about 1.2 mg; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt in the combination is from about 4.5 to about 8 mg, from about 4.5 to about 5.5 mg, preferably about 5.5 mg.

As indicated above, the invention also relates to a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof and optionally one or more pharmaceutically acceptable diluents, excipients or carriers.

The term “pharmaceutically acceptable diluent, excipient or carrier” as used herein refers to a carrier or excipient or diluent that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

### Formulations and Modes of Administration

The formulation and route of administration chosen may be tailored to the individual subject, the nature of the condition to be treated in the subject, and generally, the judgment of the attending practitioner.

The pharmaceutical compositions or combined preparations of the invention may be administered in either single or multiple doses by any of the accepted modes of administration of agents having similar utilities, including rectal, buccal, intranasal, transmucosal, transdermal, by intra-arterial injection, intravenously, intraperitoneally, parenterally, intramuscularly, subcutaneously, orally, topically, as e.g. an inhalant via pulmonary adminstration, or via an impregnated or coated device such as a stent, for example, or an artery-inserted cylindrical polymer.

One mode for administration is administration by injection, preferably intravenous administration by injection. The forms in which the compound of formula I or a pharmaceutically acceptable salt thereof and/or cyclo(-Tyr-His-Ala-Cys-Ser-Ala- ° Pro-Dab-Arg-Tyr-Cys-Tyr-Gln-Lys-Pro-Pro-) having a disulfide bond between Cys4 and Cys11 or a pharmaceutically acceptable salt thereof, may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline may also conventionally be used for injection, preferably physiologically compatible buffers such as Hank ̆s solution, Ringer ̆s s solution, or physiological saline buffer are used as aqueous solutions. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. In certain embodiments, for parenteral administration, sterile injectable solutions are prepared containing a therapeutically effective amount, e.g., 0.1 to 1000 mg, of the compound of formula I or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

A pharmaceutical combination according to the invention is, preferably, suitable for injection, e.g. subcutaneous, intravenous, intramuscular, intrathecal or intraperitoneal injection, more preferably suitable for intravenous injection, and usually comprises a therapeutically effective amount of the active ingredients and one or more suitable pharmaceutically acceptable diluent, excipient or carrier. Thus in a preferred embodiment the pharmaceutical combination is administered to the subject intravenously. i.e. the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof are administered to the subject intravenously.

Pharmaceutical compositions or combined preparations in separate form comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof may be manufactured by means of conventional mixing, dissolving, granulating, coated tablet-making, levigating, emulsifying, encapsulating, entrapping or lyophilizing processes.

Pharmaceutical compositions or combined preparations in separate form may be formulated in conventional manner using one or more physiologically acceptable carriers, diluents, excipients or auxilliaries which facilitate processing of the active ingredient into preparations which can be used pharmaceutically. Proper formulation depends upon the method of administration chosen.

For topical administration the compound of formula I or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof may be formulated as solutions, gels, ointments, creams, suspensions, etc. as are well-known in the art.

For transmucosal administration, penetrants appropriate to the barrier to be permeated are used in the formulation as known in the art.

For oral administration, the compounds can be readily formulated by combining the compound of formula I or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof with pharmaceutically acceptable carriers well known in the art. Such carriers enable the the compound of formula I or a pharmaceutically acceptable salt thereof and/or cyclo(-Tyr-His-Ala-Cys-Ser-Ala- ° Pro-Dab-Arg-Tyr-Cys-Tyr-Gln-Lys-^{D}Pro-Pro-) having a disulfide bond between Cys4 and Cys11 or a pharmaceutically acceptable salt thereof to be formulated as tablets, pills, dragees, capsules, liquids, gels, syrups, slurries, suspensions etc., for oral imzestion by a patient to be treated. For oral formulations such as, for example, powders, capsules and tablets, suitable excipients include fillers such as sugars, e.g. lactose, sucrose, mannitol and sorbitol; cellulose preparations such as maize starch, wheat starch, rice starch, potato starch, gelatin, gum tragacanth, methyl cellulose, hydroxypropylmethyl cellulose, sodium carboxymethylcellulose; granulating agents; and binding agents. If desired, desintegrating agents may be added, such as cross-linked polyvinylpyrrolidones, agar, or alginic acid or a salt thereof, such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. In addition, flavoring agents, preservatives, coloring agents and the like may be added.

For buccal administration, the composition may take the form of tablets, lozenges, etc. formulated as usual.

For administration by inhalation, the compound of formula I or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof are conveniently delivered in form of an aeorosol spray from pressurized packs or a nebulizer, with the use of a suitable propellant, e.g. dichlorodifluoromethane, trichlorofluromethane, carbon dioxide or another suitable gas. In the case of a pressurized aerosol the dose unit may be determined by providing a valve to deliver a metered amount. Capsules and cartridges of e.g. gelatin for use in an inhaler or insufflator may be formulated containing a powder mix of the compounds of the invention and a suitable powder base such as lactose or starch.

The compounds may also be formulated in rectal or vaginal compositions such as suppositories together with appropriate suppository bases such as cocoa butter or other glycerides.

In addition to the formulations described previously, the compound of formula I or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof may also be formulated as depot preparations. Such long acting formulations may be administered by implantation (e.g. subcutaneously or intramuscularly) or by intramuscular injection. For the manufacture of such depot preparations the compound of formula I or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof may be formulated with suitable polymeric or hydrophobic materials (e.g. as an emulsion in an acceptable oil) or ion exchange resins, or as sparingly soluble salts.

In addition, other pharmaceutical delivery systems may be employed such as liposomes and emulsions well known in the art. Certain organic solvents such as dimethylsulfoxide may also be employed. Additionally, the compound of formula I or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof may be delivered using a sustained-release system, such as semipermeable matrices of solid polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic agent, additional strategies for protein stabilization may be employed.

### Using the Combinations of the Invention to Treat Cancer

According to a second aspect the present invention provides a pharmaceutical combination as described herein, for use as a medicament.

According to a third aspect the present invention provides a pharmaceutical combination as described herein, for use in a method for the prevention, delay of progression or treatment of cancer in a subject, preferably for use in a method for the delay of progression or treatment of cancer in a subject, more preferably for use in a method for the treatment of cancer in a subject.

Also provided is the use of a pharmaceutical combination as described herein for the manufacture of a medicament for the prevention, delay of progression or treatment of cancer in a subject preferably for the manufacture of a medicament for the delay of progression or treatment of cancer in a subject, more preferably for the manufacture of a medicament for the treatment of cancer in a subject.

Also provided is the use of a pharmaceutical combination as described herein for the prevention, delay of progression or treatment of cancer in a subject, preferably for the delay of progression or treatment of cancer in a subject, more preferably for the treatment of cancer in a subject.

Also provided is a method for the prevention, delay of progression or treatment of cancer in a subject, preferably a method for the delay of progression or treatment of cancer in a subject, more preferably a method for the treatment of cancer in a subject, comprising administering to said subject a pharmaceutical combination as described herein e.g. administering to said subject a therapeutically effective amount of a pharmaceutical combination as described herein.

As used herein, the term “prevention”/“preventing” e.g. preventive treatments comprise prophylactic treatments. In preventive applications, the pharmaceutical combination of the invention is administered to a subject suspected of having, or at risk for developing cancer.

As used herein, the term “delay of progression”/“delaying of progression” means increasing the time to appearance of a symptom of a cancer or a mark associated with a cancer or slowing the increase in severity of a symptom of a cancer. Further, “delay of progression” as used herein includes reversing or inhibition of disease progression. “Inhibition” of disease progression or disease complication in a subject means preventing or reducing the disease progression and/or disease complication in the subject.

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof; and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

In therapeutic applications, the pharmaceutical combination is usually administered to a subject such as a patient already suffering from cancer, in an amount sufficient to cure or at least partially arrest the symptoms of the disease. Amounts effective for this use will depend on the severity and course of the disease, previous therapy, the subject's health status and response to the drugs, and the judgment of the treating physician.

In the case wherein the subject's condition does not improve, the pharmaceutical combination of the invention may be administered chronically, which is, for an extended period of time, including throughout the duration of the subject's life in order to ameliorate or otherwise control or limit the symptoms of the subject's disease or condition. In the case wherein the subject's status does improve, the pharmaceutical combination may be administered continuously; alternatively, the dose of drugs being administered may be temporarily reduced or temporarily suspended for a certain length of time (i.e., a “drug holiday”).

Once improvement of the patient's condition has occurred, a maintenance dose of the pharmaceutical combination of the invention is administered if necessary. Subsequently, the dosage or the frequency of administration, or both, is optionally reduced, as a function of the symptoms, to a level at which the improved disease is retained.

In one embodiment of the invention, there is provided a pharmaceutical combination according to the invention, for use in a method for the prevention, delay of progression or treatment of cancer in a subject, preferably for use in a method for the delay of progression or treatment of cancer in a subject, more preferably for use in a method for the treatment of cancer in a subject, wherein the cancer is selected from the group consisting of breast cancer, metastatic breast cancer, and relapsed metastatic breast cancer, preferably selected from the group consisting of metastatic breast cancer and relapsed metastatic breast cancer, more preferably wherein the cancer is relapsed metastatic breast cancer. Usually the cancer to be treated by the method for the prevention, delay of progression or treatment of cancer of the present invention expresses CXCR4. Methods of assessment of CXCR4 expression of a cancer have been reported in the scientific literature and are known to the skilled person. CXCR4 expression of a cancer can be assessed by e,g, immunohistochemistry on tumour tissue (archival primary tumour, metastatic tissue, or from a fresh biopsy). Any level of expression of CXCR4 of the cancer to be treated by the method of the present invention is considered as cancer expressing CXCR4 in the context of the invention.

Also provided is the use of a pharmaceutical combination as described herein for the manufacture of a medicament for the prevention, delay of progression or treatment of cancer in a subject, preferably for the manufacture of a medicament for the delay of progression or treatment of cancer in a subject, more preferably for the manufacture of a medicament for the treatment of cancer in a subject, wherein the cancer is selected from the group consisting of breast cancer, metastatic breast cancer, and relapsed metastatic breast cancer, preferably selected from the group consisting of metastatic breast cancer and relapsed metastatic breast cancer, more preferably wherein the cancer is relapsed metastatic breast cancer.

Also provided is the use of a pharmaceutical combination as described herein for the prevention, delay of progression or treatment of cancer in a subject, preferably for the delay of progression or treatment of cancer in a subject, more preferably for the treatment of cancer in a subject, wherein the cancer is selected from the group consisting of breast cancer, metastatic breast cancer, and relapsed metastatic breast cancer, preferably selected from the group consisting of metastatic breast cancer and relapsed metastatic breast cancer, more preferably wherein the cancer is relapsed metastatic breast cancer.

Also provided is a method for the prevention, delay of progression or treatment of cancer in a subject, preferably a method for the delay of progression or treatment of cancer in a subject, more preferably a method for the treatment of cancer in a subject, comprising administering to said subject a pharmaceutical combination as described herein e.g. administering to said subject a therapeutically effective amount of a pharmaceutical combination as described herein, wherein the cancer is selected from the group consisting of breast cancer, metastatic breast cancer, relapsed metastatic breast cancer, preferably selected from the group consisting of metastatic breast cancer and relapsed metastatic breast cancer, more preferably wherein the cancer is relapsed metastatic breast cancer.

In a preferred embodiment the cancer is selected from the group consisting of HER2-negative estrogen receptor negative progesterone receptor negative (HER2−ER−PR−) breast cancer, HER2-negative estrogen receptor negative progesterone receptor negative (HER2−ER−PR−) metastatic breast cancer, HER2-negative estrogen receptor negative progesterone receptor negative (HER2−ER−PR−) relapsed metastatic breast cancer, HER2-negative estrogen receptor positive progesterone receptor negative (HER2−ER+PR−) breast cancer, HER2-negative estrogen receptor positive progesterone receptor negative (HER2−ER+PR−) metastatic breast cancer, HER2-negative estrogen receptor positive progesterone receptor negative (HER2−ER+PR−) relapsed metastatic breast cancer, HER2-negative estrogen receptor negative progesterone receptor positive (HER2−ER−PR+) breast cancer, HER2-negative estrogen receptor negative progesterone receptor positive (HER2−ER−PR+) metastatic breast cancer, HER2-negative estrogen receptor negative progesterone receptor positive (HER2−ER−PR+) relapsed metastatic breast cancer, HER2-negative estrogen receptor positive progesterone receptor positive (HER2−ER+PR+) breast cancer, HER2-negative estrogen receptor positive progesterone receptor positive (HER2−ER+PR+) metastatic breast cancer, HER2-negative estrogen receptor positive progesterone receptor positive (HER2−ER+PR+) relapsed metastatic breast cancer, HER2-positive estrogen receptor negative progesterone receptor negative (HER2+ER−PR−) breast cancer, HER2-positive estrogen receptor negative progesterone receptor negative (HER2+ER−PR−) metastatic breast cancer, HER2-positive estrogen receptor negative progesterone receptor negative (HER2+ER−PR−) relapsed metastatic breast cancer, HER2-positive estrogen receptor positive progesterone receptor negative (HER2+ER+PR−) breast cancer, HER2-positive estrogen receptor positive progesterone receptor negative (HER2+ER+PR−) metastatic breast cancer, HER2-positive estrogen receptor positive progesterone receptor negative (HER2+ER+PR−) relapsed metastatic breast cancer, HER2-positive estrogen receptor negative progesterone receptor positive (HER2+ER−PR+) breast cancer, HER2-positive estrogen receptor negative progesterone receptor positive (HER2+ER−PR+) metastatic breast cancer, HER2-positive estrogen receptor negative progesterone receptor positive (HER2+ER−PR+) relapsed metastatic breast cancer, HER2-positive estrogen receptor positive progesterone receptor positive (HER2+ER+PR+) breast cancer, HER2-positive estrogen receptor positive progesterone receptor positive (HER2+ER+PR+) metastatic breast cancer, and HER2-positive estrogen receptor positive progesterone receptor positive (HER2+ER+PR+) relapsed metastatic breast cancer.

In a more preferred embodiment the cancer is selected from the group consisting of HER2-negative estrogen receptor negative progesterone receptor negative (HER2−ER−PR−) breast cancer, HER2-negative estrogen receptor negative progesterone receptor negative (HER2−ER−PR−) metastatic breast cancer, HER2-negative estrogen receptor negative progesterone receptor negative (HER2−ER−PR−) relapsed metastatic breast cancer, HER2-negative estrogen receptor positive progesterone receptor negative (HER2−ER+PR−) breast cancer, HER2-negative estrogen receptor positive progesterone receptor negative (HER2−ER+PR−) metastatic breast cancer, HER2-negative estrogen receptor positive progesterone receptor negative (HER2−ER+PR−) relapsed metastatic breast cancer, HER2-negative estrogen receptor negative progesterone receptor positive (HER2−ER−PR+) breast cancer, HER2-negative estrogen receptor negative progesterone receptor positive (HER2−ER−PR+) metastatic breast cancer, HER2-negative estrogen receptor negative progesterone receptor positive (HER2−ER−PR+) relapsed metastatic breast cancer, HER2-negative estrogen receptor positive progesterone receptor positive (HER2−ER+PR+) breast cancer. HER2-negative estrogen receptor positive progesterone receptor positive (HER2−ER+PR+) metastatic breast cancer, and HER2-negative estrogen receptor positive progesterone receptor positive (HER2−ER+PR+) relapsed metastatic breast cancer.

In an even more preferred embodiment the cancer is selected from the group consisting of HER2-negative estrogen receptor negative progesterone receptor negative (HER2−ER−PR−) metastatic breast cancer, HER2-negative estrogen receptor negative progesterone receptor negative (HER2−ER−PR−) relapsed metastatic breast cancer, HER2-negative estrogen receptor positive progesterone receptor negative (HER2−ER+PR−) metastatic breast cancer, HER2-negative estrogen receptor positive progesterone receptor negative (HER2−ER+PR−) relapsed metastatic breast cancer, HER2-negative estrogen receptor negative progesterone receptor positive (HER2−ER-PR+) metastatic breast cancer, HER2-negative estrogen receptor negative progesterone receptor positive (HER2−ER−PR+) relapsed metastatic breast cancer, HER2-negative estrogen receptor positive progesterone receptor positive (HER2−ER+PR+) metastatic breast cancer, HER2-negative estrogen receptor positive progesterone receptor positive (HER2−ER+PR+) relapsed metastatic breast cancer, HER2-positive estrogen receptor negative progesterone receptor negative (HER2+ER-PR−) metastatic breast cancer, HER2-positive estrogen receptor negative progesterone receptor negative (HER2+ER−PR−) relapsed metastatic breast cancer, HER2-positive estrogen receptor positive progesterone receptor negative (HER2+ER+PR−) metastatic breast cancer, HER2-positive estrogen receptor positive progesterone receptor negative (HER2+ER+PR−) relapsed metastatic breast cancer, HER2-positive estrogen receptor negative progesterone receptor positive (HER2+ER−PR+) metastatic breast cancer, HER2-positive estrogen receptor negative progesterone receptor positive (HER2+ER−PR+) relapsed metastatic breast cancer, HER2-positive estrogen receptor positive progesterone receptor positive (HER2+ER+PR+) metastatic breast cancer, and HER2-positive estrogen receptor positive progesterone receptor positive (HER2+ER+PR+) relapsed metastatic breast cancer.

In a particular preferred embodiment the cancer is selected from the group consisting of HER2-negative estrogen receptor negative progesterone receptor negative (HER2−ER−PR−) metastatic breast cancer, HER2-negative estrogen receptor negative progesterone receptor negative (HER2−ER−PR−) relapsed metastatic breast cancer, HER2-negative estrogen receptor positive progesterone receptor negative (HER2−ER+PR−) metastatic breast cancer, HER2-negative estrogen receptor positive progesterone receptor negative (HER2−ER+PR−) relapsed metastatic breast cancer, HER2-negative estrogen receptor negative progesterone receptor positive (HER2−ER−PR+) metastatic breast cancer, HER2-negative estrogen receptor negative progesterone receptor positive (HER2−ER−PR+) relapsed metastatic breast cancer, HER2-negative estrogen receptor positive progesterone receptor positive (HER2−ER+PR+) metastatic breast cancer, and HER2-negative estrogen receptor positive progesterone receptor positive (HER2−ER+PR+) relapsed metastatic breast cancer.

In a more particular preferred embodiment the cancer is selected from the group consisting of HER2-negative estrogen receptor negative progesterone receptor negative (HER2−ER−PR−) relapsed metastatic breast cancer,
HER2-negative estrogen receptor positive progesterone receptor negative (HER2−ER+PR−) relapsed metastatic breast cancer,
HER2-negative estrogen receptor negative progesterone receptor positive (HER2−ER−PR+) relapsed metastatic breast cancer,
HER2-negative estrogen receptor positive progesterone receptor positive (HER2−ER+PR+) relapsed metastatic breast cancer,
HER2-positive estrogen receptor negative progesterone receptor negative (HER2+ER-PR−) relapsed metastatic breast cancer,
HER2-positive estrogen receptor positive progesterone receptor negative (HER2+ER+PR−) relapsed metastatic breast cancer,
HER2-positive estrogen receptor negative progesterone receptor positive (HER2+ER−PR+) relapsed metastatic breast cancer,
and HER2-positive estrogen receptor positive progesterone receptor positive (HER2+ER+PR+) relapsed metastatic breast cancer.

In an even more particular preferred embodiment the cancer is selected from the group consisting of HER2-negative estrogen receptor negative progesterone receptor negative (HER2−ER−PR−) relapsed metastatic breast cancer,
HER2-negative estrogen receptor positive progesterone receptor negative (HER2−ER+PR−) relapsed metastatic breast cancer,
HER2-negative estrogen receptor negative progesterone receptor positive (HER2−ER−PR+) relapsed metastatic breast cancer,
HER2-negative estrogen receptor positive progesterone receptor positive (HER2−ER+PR+) relapsed metastatic breast cancer.

In the most particular preferred embodiment the cancer is selected from the group consisting of HER2-negative estrogen receptor positive progesterone receptor negative (HER2−ER+PR−) relapsed metastatic breast cancer and HER2-negative estrogen receptor positive progesterone receptor positive (HER2−ER+PR+) relapsed metastatic breast cancer. Thus there is preferably provided a pharmaceutical combination according to the invention, for use in a method for the prevention, delay of progression or treatment of cancer wherein the cancer is HER2-negative relapsed metastatic breast cancer, more preferably wherein the cancer is a HER2-negative relapsed metastatic breast cancer selected from the group consisting of HER2-negative estrogen receptor negative progesterone receptor negative (HER2−ER−PR−) relapsed metastatic breast cancer,
HER2-negative estrogen receptor positive progesterone receptor negative (HER2−ER+PR−) relapsed metastatic breast cancer,
HER2-negative estrogen receptor negative progesterone receptor positive (HER2−ER-PR+) relapsed metastatic breast cancer,
HER2-negative estrogen receptor positive progesterone receptor positive (HER2−ER+PR+) relapsed metastatic breast cancer in a subject,

even more preferably wherein the cancer is a HER2-negative relapsed metastatic breast cancer selected from the group consisting of HER2-negative estrogen receptor positive progesterone receptor negative (HER2−ER+PR−) relapsed metastatic breast cancer and HER2-negative estrogen receptor positive progesterone receptor positive (HER2−ER+PR+) relapsed metastatic breast cancer.

In one embodiment the subject who has cancer is (i) refractory to at least one chemotherapy treatment, or (ii) is in relapse after treatment with chemotherapy, or a combination thereof. In some embodiments, the subject is refractory to at least two, at least three, or at least four anti-cancer therapy (including, for example, standard or experimental chemotherapies). A subject who is refractory to at least one anti-cancer therapy and/or is in relapse after treatment with at least one anti-cancer therapy, as described above, may have undergone one or more prior therapies. In some embodiments, such subjects have undergone one, two, three, or four, or five, or at least one, at least two, at least three, at least four, or at least five, or between one and ten, between one and nine, between one and eight, between one and seven, between one and six, between one and five, or between one and four, or between one and three, between four and six or between seven and ten anti-cancer therapies prior to treatment using the methods described herein (e.g., prior to the administration of the compound of formula I, or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof).

### Dosing Regimen

The dosing regimen of the compound of formula I, or a pharmaceutically acceptable salt thereof, in combination with cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, in the methods provided herein may vary depending upon the indication, route of administration, and severity of the condition, for example. Depending on the route of administration, a suitable dose can be calculated according to body weight, body surface area, or organ size. The final dosing regimen can be determined by the attending physician in view of good medical practice, considering various factors that modify the action of drugs, e.g., the specific activity of the compound, the identity and severity of the disease state, the responsiveness of the patient, the age, condition, body weight, sex, and diet of the patient, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

The amount, e.g. the therapeutically effective amount of the compound of formula I, or a pharmaceutically acceptable salt thereof, may be provided in a single dose or multiple doses to achieve the desired treatment endpoint.

The frequency of dosing will depend on the pharmacokinetic parameters of the compound administered, the route of administration, and the particular disease treated. The dose and frequency of dosing may also depend on pharmacokinetic and pharmacodynamic, as well as toxicity and therapeutic efficiency data. For example, pharmacokinetic and pharmacodynamic information about the compound of formula I, or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, can be collected through preclinical in vitro and in vivo studies, later confirmed in humans during the course of clinical trials. Thus, for the ccompound of formula I, or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, used in the methods provided herein, a therapeutically effective dose can be estimated initially from biochemical and/or cell-based assays. Then, dosage can be formulated in animal models to achieve a desirable circulating concentration range. As human studies are conducted further information will emerge regarding the appropriate dosage levels and duration of treatment for various diseases and conditions.

Toxicity and therapeutic efficacy of the compound of formula I, or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the “therapeutic index”, which typically is expressed as the ratio LD50/ED50. Compounds that exhibit large therapeutic indices, i.e. the toxic doses are substantially higher than the effective doses, are preferred. The data obtained from such cell culture assays and additional animal studies can be used in formulating a range of dosage for human use. The doses of such compounds lies preferably within a range of circulating concentrations that include the ED50 with little or no toxicity.

The compound of formula I, or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, may be administered to the subject (e.g. a human) within minutes or hours. In some embodiments, the compound of formula I, or a pharmaceutically acceptable salt thereof and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, may be administered to the subject (e.g. a human) over about 1 to about 240 minutes, over about 1 to about 180 minutes, or over about 5 to about 150 minutes, or over about 5 to about 120 minutes, or over about 1 to 60 minutes, or over about 1 to 10 minutes, or over about 5 minutes.

In one embodiment the compound of formula I or a pharmaceutically acceptable salt thereof is administered to the subject (e.g. a human) usually over about 1 to about 60 minutes, preferably over about 2 to about 30 minutes, more preferably over about 2 to about 10 minutes, most preferably over about 5 minutes.

In one embodiment cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof is administered to the subject (e.g. a human) usually over about 1 to about 240 minutes, preferably over about 1 to about 180 minutes, or more preferably over about 60 to about 150 minutes, most preferably over about 120 minutes.

In one embodiment the compound of formula I or a pharmaceutically acceptable salt thereof is administered to the subject (e.g. a human) after the end of the administration of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof. In a preferred embodiment the compound of formula I or a pharmaceutically acceptable salt thereof is usually administered about 15 to about 240 minutes, about 15 to about 120 minutes, preferably about 20 to about 60 minutes, more preferably about 20 to about 30 minutes, even more preferably about 25 minutes after the end of the administration of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof.

An exemplary treatment regime entails administration once daily, twice daily, three times daily, every day, every second day, every third day, every fourth day, every fifth day, every sixth day, twice per week, once per week. The combination of the invention is usually administered on multiple occasions. Intervals between single dosages can be, for example, less than a day, a day, two days, three days, four days, five days, six days or a week. The combination of the invention may be given as a continous uninterrupted treatment. The combination of the invention may also be given in a regime in which the subject receives cycles of treatment (administration cycles) interrupted by a drug holiday or period of non-treatment. Thus, the combination of the invention may be administered according to the selected intervals above for a continuous period of one week or a part thereof, for two weeks, for three weeks for four weeks, for five weeks or for six weeks and then stopped for a period of one week, or a part thereof, for two weeks, for three weeks, for four weeks, for five weeks, or for six weeks or for even more weeks. The combination of the treatment interval and the non-treatment interval is called a cycle. The cycle may be repeated one or more times. Two or more different cycles may be used in combination for repeating the treatment one or more times. A preferred administration cycle in the methods of the present invention is a period of three weeks i.e. a 21-day cycle. In one embodiment, the cycle is repeated one or more times, usually one, two, three, for, five, six, seven, eight , nine, ten, eleven, twelve, thirteen, fourteen, fifteen, sixteen, seventeen, eighteen, nineteen, twenty or twenty one times, preferably at least two, at least three, at least four, at least five, at least six seven, at least eight, at least nine, at least ten, at least eleven, at least twelve, at least thirteen, at least fourteen, at least fifteen, at least sixteen, at least seventeen, at least eighteen, at least nineteen, at least twenty or at least twenty one times, more preferably at least two times. In a particular preferred embodiment the cycle is repeated at least two times, so that the treatment comprises at least three 21-day cycles.

The administration of the pharmaceutical combination according to the invention may start with a run-in cycle e.g. with a run-in cycle followed by 21-days cycles. In one embodiment the administration of the pharmaceutical combination according to the invention starts with a run-in cycle followed by 21-days cycles. The run-in cycle may last 28 days, wherein the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, is independently administered to the subject, wherein the compound of formula I or a pharmaceutically acceptable salt thereof is administered on days 1 and 16 and wherein cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is administered on day 1, and on days 15, 16 and 17. In a preferred embodiment, the administration of the pharmaceutical combination according to the invention starts with a 21-day cycle. In this embodiment no run-in cycle is administered before the first 21-day cycle. In a more preferred embodiment, the pharmaceutical combination according to the invention is administered on days 1, 2 and 3, and on days 8, 9 and 10 of a 21-day cycle of administration.

In one preferred embodiment, the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, is independently administered to the subject, wherein the compound of formula I or a pharmaceutically acceptable salt thereof is administered on days 2 and 9 and wherein cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is administered on days 1, 2 and 3, and on days 8, 9 and 10 of a 21-day cycle of administration.

In one preferred embodiment the pharmaceutical combination of the present invention is administered for a 21-day cycle, wherein on day 2 and 9 of the 21-day cycle of administration the compound of formula I or a pharmaceutically acceptable salt thereof is administered over about 2 to about 10 minutes to the subject about 15 to about 120 minutes after the end of the administration of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof which is administered over about 1 to about 3 hours. In a more preferred embodiment the pharmaceutical combination of the present invention is administered for a 21-day cycle, wherein on day 2 and 9 of the 21-day cycle of administration the compound of formula I or a pharmaceutically acceptable salt thereof is administered over about 5 minutes to the subject about 25 minutes after the end of the administration of cyclo(-Tyr-His-Ala-Cys-Ser-Ala-^{D}Pro-Dab-Are-Tyr-Cys-Tyr-Gln-Lys-^{D}Pro-Pro-) having a disulfide bond between Cys4 and Cys11 or a pharmaceutically acceptable salt thereof which is administered over about 2 hours.

Exemplary doses of the compound of formula I or a pharmaceutically acceptable salt thereof for a human subject may be from about 0.1 to about 50 mg/m² or from about 0.1 to about 20 mg/m² or from about 0.1 to about 10 mg/m² or from about 0.5 to about 8 mg/m² or from about 0.5 to about 6 mg/m² or from about 1 to about 2 mg/m² or about 1 mg/m², about 1.2 mg/m², about 1.5 mg/m², about 2 mg/m², about 3 mg/m², about 4 mg/m², about 5 mg/m², about 6 mg/m², about 7 mg/m², about 8 mg/m² about 9 mg/m² or about 10 mg/m².

Exemplary doses of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, for a human subject may be from about 0.1 to about 50 mg/kg or from about 0.1 to about 20 mg/kg or from about 0.1 to about 10 mg/kg or from about 0.5 to about 8 mg/kg or from about 0.5 to about 6 mg/kg, or from about 1 to about 5.5 mg/kg, or from about 4.5 to about 8 mg/kg or from about 4.5 to about 5.5 mg/kg, or about 1 mg/kg, about 2 mg/kg, about 2.5 mg/kg, about 3 mg/kg, about 3.5 mg/kg, about 4 mg/kg, about 4.5 mg/kg, about 5.5 mg/kg, about 7.5 mg/kg or about 10 mg/kg.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said compound of formula I or a pharmaceutically acceptable salt thereof in the combination is from about 0.1 to about 50 mg/m² or from about 0.1 to about 20 mg/m² or from about 0.1 to about 10 mg/m² or from about 0.5 to about 8 mg/m² or from about 0.5 to about 6 mg/m² or from about 1 to about 2 mg/m²; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof in the combination is from about 0.1 to about 50 mg/kg or from about 0.1 to about 20 mg/kg or from about 0.1 to about 10 mg/kg or from about 0.5 to about 8 mg/kg or from about 0.5 to about 6 mg/kg, or from about 1 to about 5.5 mg/kg or from about 4.5 to about 8 mg/kg, or from about 4.5 to about 5.5 mg/kg.

In a preferred embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the amount of said compound of formula I or a pharmaceutically acceptable salt thereof in the combination is about 1 mg/m², about 1.2 mg/m², about 1.5 mg/m², about 2 mg/m², about 3 mg/m², about 4 mg/m², about 5 mg/m², about 6 mg/m², about 7 mg/m², about 8 mg/m², about 9 mg/m² or about 10 mg/m^{2 ;} and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof in the combination is about 1 mg/kg, about 2 mg/kg, about 2.5 mg/kg, about 3 mg/kg, about 3.5 mg/kg, about 4 mg/kg, about 4.5 mg/kg, about 5.5 mg/kg, about 7.5 mg/kg or about 10 mg/kg.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the compound of formula I or a pharmaceutically acceptable salt thereof is administered to the subject at a dose between about 0.1 and about 10 mg/m², preferably between 0.5 to about 6 mg/m², more preferably between about 1 to about 2 mg/m², even more preferably about 1.2 mg/m².

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is administered to the subject at a dose between about 0.1 and about 10 mg/kg, preferably from about 1 to about 5.5 mg/kg, more preferably from about 4.5 to about 8 mg/kg, even more preferably from about 4.5 to about 5.5 mg/kg, most preferably at a dose of about 5.5 mg/kg.

In one embodiment, the invention provides a pharmaceutical combination comprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the compound of formula I or a pharmaceutically acceptable salt thereof is administered to the subject at a dose between about 0.1 and about 10 mg/m², preferably between 0.5 to about 6 mg/m², more preferably between about 1 to about 2 mg/m², even more preferably about 1.2 mg/m² and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is administered at a dose between about 0.1 and about 10 mg/kg. preferably from about 1 to about 5.5 mg/kg, more preferably from about 4.5 to about 8 mg/kg, even more preferably from about 4.5 to about 5.5 mg/kg, most preferably at a dose of about 5.5 mg/kg.

In a particular embodiment, the invention provides a pharmaceutical combinationcomprising a compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, wherein the compound of formula I or a pharmaceutically acceptable salt thereof is administered to the subject at a dose about 1.2 mg/m² and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is administered to the subject at a dose of about 5.5 mg/kg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said compound of formula Ia in the combination is from about 0.1 to about 50 mg/m² or from about 0.1 to about 20 mg/m² or from about 0.1 to about 10 mg/m² or from about 0.5 to about 8 mg/m² or from about 0.5 to about 6 mg/m² or from about 1 to about 2 mg/m²; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt in the combination is from about 0.1 to about 50 mg/kg or from about 0.1 to about 20 mg/kg or from about 0.1 to about 10 mg/kg or from about 0.5 to about 8 mg/kg or from about 0.5 to about 6 mg/kg, or from about 1 to about 5.5 mg/kg or from about 4.5 to about 8 mg/kg, or from about 4.5 to about 5.5 mg/kg.

In a preferred embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the amount of said compound of formula Ia in the combination is about 1 mg/m², about 1.2 mg/m², about 1.5 mg/m², about 2 mg/m², about 3 mg/m², about 4 mg/m², about 5 mg/m², about 6 mg/m², about 7 mg/m², about 8 mg/m², about 9 mg/m² or about 10 mg/m²; and wherein the amount of said cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt in the combination is about 1 mg/kg, about 2 mg/kg, about 2.5 mg/kg, about 3 mg/kg, about 3.5 mg/kg, about 4 mg/kg, about 4.5 mg/kg, about 5.5 mg/kg, about 7.5 mg/kg or about 10 mg/kg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the compound of formula Ia is administered to the subject at a dose between about 0.1 and about 10 mg/m², preferably between 0.5 to about 6 mg/m², more preferably between about 1 to about 2 mg/m², even more preferably about 1.2 mg/m².

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt is administered to the subject at a dose between about 0.1 and about 10 mg/kg. preferably from about 1 to about 5.5 mg/kg, more preferably from about 4.5 to about 8 mg/kg, even more preferably from about 4.5 to about 5.5 mg/kg, most preferably at a dose of about 5.5 mg/kg.

In one embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the compound of formula Ia is administered to the subject at a dose between about 0.1 and about 10 mg/m², preferably between 0.5 to about 6 mg/m², more preferably between about 1 to about 2 mg/m², more preferably about 1.2 mg/m² and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt is administered at a dose between about 0.1 and about 10 mg/kg. preferably from about 1 to about 5.5 mg/kg, more preferably from about 4.5 to about 8 mg/kg, even more preferably from about 4.5 to about 5.5 mg/kg, most preferably at a dose of about 5.5 mg/kg.

In a particular embodiment, the invention provides a pharmaceutical combination comprising a compound of formula Ia and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt, wherein the compound of formula Ia is administered to the subject at a dose about 1.2 mg/m² and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 acetate salt is administered to the subject at a dose of about 5.5 mg/kg.

### Additonal Combination Therapies

Provided herein are also methods of treatment in which the compound of formula I, or a pharmaceutically acceptable salt thereof, in combination with cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, is administered to a subject (e.g. a human) in additional combination with one or more additional therapies. Thus, in some embodiments, the method for treating cancer in a subject (e.g. a human), comprises administering to the subject a therapeutically effective amount of a compound of formula I, or a pharmaceutically acceptable salt thereof, and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, together with one or more additional therapies, which can be useful for treating the cancer. The one or more additional therapies may involve the administration of one or more therapeutic agents, preferably therapeutic anti-cancer agents.

### Kit of Parts

A pharmaceutical combination e.g. a combined preparation (including, for example, formulations and unit dosages) comprising the compound of formula I, or a pharmaceutically acceptable salt thereof, and cyclo(-Tyr-His-Ala-Cys-Ser-Ala-^{D}Pro-Dab-Arg-Tyr-Cys-Tyr-Gln-Lys- ° Pro-Pro-) having a disulfide bond between Cys4 and Cys11, or a pharmaceutically acceptable salt thereof, can be prepared and placed in an appropriate container, and labeled for treatment of an indicated condition.

Kits of parts also are contemplated. For example, a kit can comprise unit dosage forms of the compound of formula 1, or a pharmaceutically acceptable salt thereof, and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, and a package insert containing instructions for use of the composition in treatment of a medical condition. In some embodiments, the kits comprises a unit dosage form of compound of formula I, or a pharmaceutically acceptable salt thereof, and/or cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof. The instructions for use in the kit may be for treating a cancer.

Thus in a fourth aspect the present invention provides a kit of parts comprising a first container, a second container and a package insert, wherein the first container comprises at least one dose of a medicament comprising a compound of formula I or a pharmaceutically acceptable salt thereof or a compound of formula Ia, the second container comprises at least one dose of a medicament comprising cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, and the package insert comprises instructions for treating a subject for cancer using the medicaments, wherein the cancer is selected from the group consisting of breast cancer, metastatic breast cancer, and relapsed metastatic breast cancer. Doses to be used in the kit of parts are as usually described above.

### Examples

The present examples are intended to illustrate the present invention without restricting it.

### Example 1

This open-label, single-arm, non-randomised Phase I, dose escalation trial enrolled HER2-negative (any oestrogen or progesterone receptor status) females age ℏ18 years with histologically confirmed invasive breast cancer, stage IV disease (American Joint Committee on Cancer criteria), evidence of tumour cell CXCR4 expression by immunohistochemistry on tumour tissue (archival primary tumour, metastatic tissue, or from a fresh biopsy), and at least one measurable lesion according to Response Evaluation Criteria in Solid Tumours (RECIST) 1.1 criteria. Patients had previously received 163 chemotherapy regimens for metastatic breast cancer (MBC). Patients with hormone receptor positive status must have failed at least one endocrine therapy or be considered unsuitable for endocrine therapy. Eligibility criteria also included an Eastern Cooperative Oncology Group performance status of 0 or 1. This phase I study investigates the combination of eribulin with POL6326 in relapsed metastatic breast cancer. The primary objectives are the safety, tolerability, and pharmacokinetics (PK) of this combination therapy, as well as the maximum tolerated dose (MTD) and recommended phase 2 dose (RP2D) of POL6326 when added to eribulin. Efficacy, as measured by tumor response, was additionally assessed.

### Study Design

Treatment cycles of 21 days each. Eribulin administered IV at the registered dose of 1.4 mg/m² over approximately 5 minutes on days 2 and 9 starting approximately 25 mins after end of POL6326 infusion and POL6326 over approximately 2 hours infusion on days 1, 2, 3 and 8, 9 and 10 (see Treatment scheme below in Table 1). Eribulin was administered IV as eribulin mesylate, whereas POL6326 was administered IV as acetate salt.

TABLE 1
Treatment Scheme for all treatment cycles

| | | | | | | | | | | | | | | | | | | | | | |
|---|---|---|---|---|---|---|---|---|---|---|---|---|---|---|---|---|---|---|---|---|---|
| Day | 1 | 2 | 3 | 4 | 5 | 6 | 7 | 8 | 9 | 10 | 11 | 12 | 13 | 14 | 15 | 16 | 17 | 18 | 19 | 20 | 21 |

| Eribulin | | 2 | break | | | 9 | treatment break | |
|---|---|---|---|---|---|---|---|---|
| POL6326 | 1 | 2 | 3 | break | 8 | 9 | 10 | treatment break |

### Measurement of effect

### Antitumor Effect—Solid Tumors

For the purposes of this study, patients were re-evaluated for response after every 2 cycles by CT scan.

Response and progression were evaluated in this study using the new international criteria proposed by the revised Response Evaluation Criteria in Solid Tumors (RECIST) guideline (version 1.1) [Eur J Ca 45:228-247, 2009]. Changes in the largest diameter (unidimensional measurement) of the tumor lesions and the shortest diameter in the case of malignant lymph nodes are used in the RECIST criteria.

### Disease Parameters

Measurable disease: Measurable lesions are defined as those that can be accurately measured in at least one dimension (longest diameter to be recorded) as ≥20 mm by chest x-ray, as ≥10 mm with CT scan, or >10 mm with calipers by clinical exam. All tumor measurements must be recorded in millimeters (or decimal fractions of centimeters).

It is to be noted that tumor lesions that are situated in a previously irradiated area might or might not be considered measurable. If the treating investigator thinks it appropriate to include them, the conditions under which such lesions should be considered must be defined in the protocol.

Malignant lymph nodes: To be considered pathologically enlarged and measurable, a lymph node must be ℏ15 mm in short axis when assessed by CT scan (CT scan slice thickness recommended to be no greater than 5 mm). At baseline and in follow-up, only the short axis will be measured and followed.

Non-measurable disease: All other lesions (or sites of disease), including small lesions (longest diameter <10 mm or pathological lymph nodes with ℏ10 to <15 mm short axis), are considered non-measurable disease. Bone lesions, leptomeningeal disease, ascites, pleural/pericardial effusions, lymphangitis cutis/pulmonitis, inflammatory breast disease, and abdominal masses (not followed by CT or MRI), are considered as non-measurable.

It is to be noted that cystic lesions that meet the criteria for radiographically defined simple cysts should not be considered as malignant lesions (neither measurable nor non-measurable) since they are, by definition, simple cysts.

:Cystic lesions ̆thought to represent cystic metastases can be considered as measurable lesions, if they meet the definition of measurability described above. However, if non-cystic lesions are present in the same patient, these are preferred for selection as target lesions. Target lesions: All measurable lesions up to a maximum of 2 lesions per organ and 5 lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. A sum of the diameters (longest for non-nodal lesions, short axis for nodal lesions) for all target lesions will be calculated and reported as the baseline sum diameters. If lymph nodes are to be included in the sum, then only the short axis is added into the sum. The baseline sum diameters will be used as reference to further characterize any objective tumor regression in the measurable dimension of the disease.

Non-target lesions: All other lesions (or sites of disease) including any measurable lesions over and above the 5 target lesions should be identified as non-target lesions and should also be recorded at baseline. Measurements of these lesions are not required, but the presence, absence, or in rare cases unequivocal progression of each should be noted throughout follow-up.

### Methods for Evaluation of Measurable Disease

All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination unless the lesion(s) being followed cannot be imaged but are assessable by clinical exam. The disease assessment and tumor evaluation should be performed in accordance to RECIST 1.1.

### Response Criteria

### Evaluation of Target Lesions

Complete Response (CR): Disappearance of all target lesions. Any pathological lymph nodes (whether target or non-target) must have reduction in short axis to <10 mm. Partial Response (PR): At least a 30% decrease in the sum of the diameters of target lesions, taking as reference the baseline sum diameters.

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progressions).

Stable Disease (SD): Neither sufficient shrinkage to qualify for PR nor sufficient increase to qualify for PD, taking as reference the smallest sum diameters while on study.

### Evaluation of Non-Target Lesions

Complete Response (CR): Disappearance of all non-target lesions and normalization of tumor marker level. All lymph nodes must be non-pathological in size (<10 mm short axis).

It is to be noted that if tumor markers are initially above the upper normal limit, they must normalize for a patient to be considered in complete clinical response.

Non-CR/Non-PD: Persistence of one or more non-target lesion(s) and/or maintenance of tumor marker level above the normal limits.

Progressive Disease (PD): Appearance of one or more new lesions and/or unequivocal progression of existing non-target lesions. Unequivocal progression should not normally trump target lesion status. It must be representative of overall disease status change, not a single lesion increase.

Although a clear progression of \grave{ }non-target_lesions only is exceptional, the opinion of the treating physician should prevail in such circumstances, and the progression status should be confirmed at a later time by the review panel (or Principal investigator).

### Evaluation of Best Overall Response

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

TABLE 2
Response for Patients with Measurable Disease (i.e., Target Disease)

| | | | | Best Overall Response |
|---|---|---|---|---|
| Target | Non-Target | New | Overall | when Confirmation |
| Lesions | Lesions | Lesions | Response | is Required* |
| CR | CR | No | CR | >4 wks. Confirmation** |
| CR | Non-CR/ | No | PR | >4 wks. Confirmation** |
| | Non-PD | | | |
| CR | Not evaluated | No | PR | |
| PR | Non-CR/ | No | PR | |
| | Non-PD/ | | | |
| | not evaluated | | | |
| SD | Non-CR/ | No | SD | Documented at least once |
| | Non-PD/ | | | >4 wks. from baseline** |
| | not evaluated | | | |
| PD | Any | Yes or No | PD | no prior SD, PR or CR |
| Any | PD*** | Yes or No | PD | |
| Any | Any | Yes | PD | |

*See RECIST 1.1 manuscript for further details on what is evidence of a new lesion.
**Only for non-randomized trials with response as primary endpoint.
***In exceptional circumstances, unequivocal progression in non-target lesions may be accepted as disease progression.
Note:
Patients with a global deterioration of health status requiring discontinuation of treatment without objective evidence of disease progression at that time should be reported as \grave{ }symptomatic deterioration_. Every effort should be made to document the objective progression even afterdiscontinuation of treatment.

TABLE 3
Response for Patients with Non-Measurable Disease
(i.e., Non-Target Disease)

| Non-Target Lesions | New Lesions | Overall Response |
|---|---|---|
| CR | No | CR |
| Non-CR/non-PD | No | Non-CR/non-PD* |
| Not all evaluated | No | not evaluated |
| Unequivocal PD | Yes or No | PD |
| Any | Yes | PD |

*Non-CR/non-PD - is preferred over: stable disease - for non-target disease since SD is increasingly used as an endpoint for assessment of efficacy in some trials so to assign this category when no lesions can be measured is not advised

### Duration of Response

Duration of overall response: The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

Duration of stable disease: Stable disease is measured from the start of the treatment until the criteria for progression are met, taking as reference the smallest measurements recorded since the treatment started, including the baseline measurements.

### Progression-Free Survival

PFS is defined as the duration of time from start of treatment to time of progression or death, whichever occurs first.

TABLE 4
Response rates from dose escalation and dose expansion cohorts
Eribulin 1.4 mg/sqm + POL6326 dose as indicated

| | Dose | | Cohort 11 + | | Cohort 11 + | |
|---|---|---|---|---|---|---|
| | escalation | | expansion | | expansion | |
| | cohorts | | cohort | | cohort | |
| | 1-4 mg/kg | | 5.5 mg/kg | | 5.5 mg/kg | |
| Response | n = 18è | | n = 24** | | n = 24*** | |
| CR | 0 | | 0 | | 0 | |
| PR | 6 | (33%) | 9^{#} | (38%) | 9 | (38%) |
| SD | 8 | (44%) | 10 | (42%) | 16 | (67%) |
| SD ℏ 6 months | 1 | (1%) | 4 | (17%) | 6 | (25%) |
| PD | 3 | (16%) | 4 | (17%) | 4 | (17%) |
| Not evaluable | 1* | (6%) | 1* | (4%) | 1* | (4%) |
| ORR | 6 | (33%) | 9^{#} | (38%) | 9^{#} | (38%) |
| CBR | 7 | (39%) | 13 | (54%) | 15 | (63%) |

ORR = objective response rate = CR + PR
CBR = clinical benefit rate = CR + PR + SD ≥ 6 months
*discontinued end of cycle 1 or deceased
è 1/18 still on treatment
**10/24 patients still on treatment
***3/24 patients still on treatment; date of data cut-off: 6 month after date of data cut-off of**
^{#}7/9 are confirmed by a subsequent CT scan after 2 cycles; 1 PR still not confirmed (time point for 2nd CT scan not yet reached); 1 PR not confirmed because pt was in PD at the subsequent CT scan.
NOTE:
protocol did not request confirmation after 4 weeks as usually done for clinical studies with efficacy as primary endpoint- in this study efficacy is NOT a primary endpoint; so confirmation can only we obtained after 6 weeks when regular CT scan/tumor assessment is done

TABLE 5
Response rates from dose escalation cohorts
Dose escalation cohorts
1-4 mg/kg
n = 18

| | HER2−/ER−/PR− | | | |
|---|---|---|---|---|
| | (TNBC) | HER2−/ER+/PR+ | | HER2−/ER+/PR− |
| Response | n = 5 | n = 8 | | n = 5 |
| CR | 0 | 0 | | 0 |
| PR | 1 (20%) | 4 | (50%) | 1 (20%) |
| SD | 4 (80%) | 3 | (38%) | 1 (20%) |
| PD | 0 | 0 | | 3 (60%) |
| Not evaluable | 0 | 1* | (13%) | 0 |
| ORR | 1 (20%) | 4 | (50%) | 1 (20%) |
| CBR | 1 (20%) | 5 | (63%) | 1 (20%) |

*discontinued end of cycle 1 or deceased
NOTE:
There were no patients HER2−/ER−/PR+

TABLE 6
Best tumor responses (dose escalation cohorts)

| | POL6326 Dose | | Number of |
|---|---|---|---|
| Cohort # | (mg/kg) | Best Tumor Responseè | Cycles |
| 1 | 1.0 | 1/3 SD (cycle 4) | 4, 6è, 1 |
| | | 1/3 PR (cycle 2, 4) | |
| | | 1/3 death | |
| 5 | 2.0 | 2/3 SD (cycle 2, 4) | 4, 6, 8è |
| | | 1/3 PR (cycle 2, 4, 6) | |
| 6 | 2.5 | (1/3 PD cycle 2) | 2, 4, 10è |
| | | 1/3 SD (cycle 2) | |
| | | 1/3 PR (cycle 6, 8, 10) | |
| 7 | 3.0 | 2/3 SD (cycle 2) | 4, 4, 4è |
| | | 1/3 PR (cycle 2) | |
| 8 | 3.5 | (1/3 PD cycle 2) | 2, 4, 8è |
| | | 1/3 SD (cycle 2) | |
| | | 1/3 PR (cycle 6) | |
| 9 | 4.0 | (1/3 PD cycle 2) | 2, 7è, 18* |
| | | 1/3 PR (cycle 2, 4, 6) | |
| | | 1/3 SD (cycle 2, 4, 6, 8, 10, 12, 14) | |

*Patient (Pt) still on treatment
è Number of cycles of patients having shown PR

TABLE 7
Responses from cohort 11 + expansion cohort, 10/24 patients still on treatment
Dose expansion cohort
5.5 mg/kg
n = 24*

| | HER2−/ER−/PR− | | | | |
|---|---|---|---|---|---|
| | (TNBC) | HER2−/ER+/PR+ | HER2−/ER+/PR− | | HER2−/ER−/PR+ |
| Response | n = 3 | n = 13 | n = 5 | | n = 2 |
| CR | 0 | 0 | 0 | | 0 |
| PR | 0 | 5 (38%) | 3 | (60%) | 0 |
| SD | 2 (67%) | 6 (46%) | 1 | (20%) | 1 (50%) |
| PD | 1 (33%) | 2 (15%) | 0 | | 1 (50%) |
| Not evaluable | 0 | 0 | 1** | (20%) | 0) |
| ORR | 0 | 5 (38%) | 3 | (60%) | 0 |
| CBR | 1 (33%) | 8 (62%) | 3 | (60%) | 0 |

*ER/PR status unknown for 1 Pt in PR
**discontinued end of cycle 1 or deceased

TABLE 8
Patients with best tumor responses (cohort 11 + expansion cohort)

| | Best Tumor | Number of | Best Tumor | Number of |
|---|---|---|---|---|
| Pt # | Response¹ | Cycles¹ | Response² | Cycles² |
| 1 | SD | 8 | | |
| 2 | SD | 14 | | |
| 3 | SD | 5 | | |
| 4 | na | 2 | | |
| 5 | na | 2 | | |
| 6 | SD | 8 | | |
| 7 | PR (cycle 6, 8) | 10* | PR (cycle 6, 8, 10) | 12 |
| 8 | PR (cycle 8, 10) | 12 | | |
| 9 | SD | 12* | SD | 14 |
| 10 | PR (cycle 2,4) | 6 | | |
| 11 | SD | 10* | SD | 17** |
| 12 | SD | 4 | | |
| 13 | SD | 10* | SD | 16 |
| 14 | SD | 4 | | |
| 15 | PR (cycle 6, 8) | 10* | PR (cycle 6, 8, 10, 12) | 13** |
| 16 | na | 2 | | |
| 17 | PR (cycle 4, 6) | 8* | PR (cycle 4, 6, 8) | 10 |
| 18 | SD | 6* | SD | 8 |
| 19 | PR (cycle 2, 4) | 6* | PR (cycle 2, 4, 6, 8) | 10 |
| 20 | deceased cycle 1 | 1 | | |
| 21 | PR (cycle 2, 4, 6) | 7* | PR (cycle 2, 4, 6) | 8 |
| 22 | na | 2 | | |
| 23 | PR (cycle 6) | 7* | PR (cycle 6, 8, 10, 12) | 12** |
| 24 | PR (cycle 4) | 6 | | |

na = Pt was in PD at first tumor assessment;
*Pt still on treatment
**Pt still on treatment
¹10/24 patients still on treatment
²3/24 patients still on treatment; date of data cut-off: 6 month after date of data cut-off of¹

### Discussion

In this Phase I trial, which included an Expanded Cohort at the highest balixafortide dose (5.5 mg/kg), we show that balixafortide +eribulin in patients with HER2-negative MBC is well-tolerated and has encouraging signs of activity. In this first trial (to our knowledge) to investigate a CXCR4 antagonist in breast cancer, the safety and tolerability of balixafortide+eribulin appeared comparable to published data on either eribulin or balixafortide monotherapy. In this trial, most treatment emergent adverse events (TEAEs, even with balixafortide 5.5 mg/kg) were similar to those reported in other studies for eribulin alone: G3/4 neutropenia 41% for balixafortide+eribulin in this trial (45 [symbol] 64% reported for eribulin alone in the literature), G3/4 leukopenia 9% for balixafortide+eribulin (14 [symbol] 18% reported for eribulin alone in the literature), G3/4 peripheral neuropathy 3.6% for balixafortide+eribulin (568% reported for eribulin alone in the literature), neuropathy of any grade 39% for balixafortide+eribulin (27 [symbol] 35% reported for eribulin alone in the literature), and no evidence that these were associated with increasing balixafortide doses. The incidence of febrile neutropenia 11% for balixafortide+eribulin (2 [symbol] 6% reported for eribulin alone in the literature), though numerically higher in this trial, did not appear to be dose-related.

Balixafortide-related infusion-related reactions (IRRs) were expected; the majority were low grade and most frequent during Day 1, Cycle 1. Such reactions are typical of peptide-based drugs and were well managed by slowing the infusion rate and providing prophylaxis with an H₁ antagonist.

Although serious adverse events (SAEs) were reported, the incidence of individual SAEs was low and no trend was observed. The rate of fatal AEs (3.6%) was comparable to the rate reported for eribulin monotherapy in published studies (4 [symbol] 4.8%) for previously treated MBC.

Although numbers were small, there appeared to be no overlapping toxicities or accentuation of known eribulin toxicities with balixafortide. In contrast, other CXCR4 antagonists have shown increased haematological risks as monotherapy and in combination with chemotherapy: LY2510924 monotherapy reported abnormal neutrophil count as a DLT; LY2510924 +carboplatin+etoposide in small cell lung cancer exacerbated neutropenia, leucopenia and anaemia compared to chemotherapy alone (Galsky M D, Vogelzang N J, Conkling P, et al. A phase I trial of LY2510924, a CXCR4 peptide antagonist, in patients with advanced cancer. Clin Cancer Res 2014; 20(13): 3581-8; Salgia R, Stine J R, Weaver R W, et al. A randomized phase II study of LY2510924 and carboplatin/etoposide versus carboplatin/etoposide in extensive-disease small cell lung cancer. Lung Cancer 2017; 105: 7-13). There was also no evidence of efficacy at the dose investigated (Salgia R, Stine J R, Weaver R W, et al. A randomized phase II study of LY2510924 and carboplatin/etoposide versus carboplatin/etoposide in extensive-disease small cell lung cancer. Lung Cancer 2017; 105: 7-13).

Although based on inter-trial comparisons, the ORR (38%) and CBR (63%) reported for balixafortide +eribulin in the Expanded Cohort were numerically much higher than those reported for eribulin alone in similar MBC populations (Cortes J, O'Shaughnessy J, Loesch D, et al. Eribulin monotherapy versus treatment of physician's choice in patients with metastatic breast cancer (EMBRACE): a phase 3 open-label randomised study. Lancet 2011; 377(9769): 914-23; Kaufman P A, Awada A, Twelves C, et al. Phase III open-label randomized study of eribulin mesylate versus capecitabine in patients with locally advanced or metastatic breast cancer previously treated with an anthracycline and a taxane. Journal of clinical oncology: official journal of the American Society of Clinical Oncology 2015; 33(6): 594-601; Cortes J, Vandat L, Blum J L, et al. Phase II study of the halichondrin B anolog eribulin mesylate in patients with locally advanced or metastatic breast cancer previously treated with an anthracycline, a taxane and capecitabine. Journal of clinical oncology: official journal of the American Society of Clinical Oncology 2010; 28(25): 3922-8; Vandat L T, Pruitt B, Fabian C J, et al. Phase II study of eribulin mesylate, a halichondrin B anolog, in patients with metastatic breast cancer previously treated with an anthracycline and a taxane Journal of clinical oncology: official journal of the American Society of Clinical Oncology 2009; 27(18): 2954-61). Moreover, all parameters of anti-tumour activity were increased in the Expanded Cohort where the highest dose of balixafortide was employed suggesting a dose response. Responses were observed regardless of CXCR4 expression level or the line of therapy for MBC at baseline, and were numerically higher in HR positive patients. In two Phase II studies with eribulin alone (Vandat et al 2009 and Cortes et al 2010, referenced above), the ORR were 11.5% and 9% and the CBR were 17.2% and 17.1%, respectively. In two eribulin-based, randomised, Phase III trials, the group of patients treated with eribulin had an ORR of 12% and 11% (Cortes J, O'Shaughnessy J, Loesch D, et al. Eribulin monotherapy versus treatment of physician's choice in patients with metastatic breast cancer (EMBRACE): a phase 3 open-label randomised study. Lancet 2011; 377(9769): 914-23; Kaufman P A, Awada A, Twelves C, et al. Phase III open-label randomized study of eribulin mesylate versus capecitabine in patients with locally advanced or metastatic breast cancer previously treated with an anthracycline and a taxane. Journal of clinical oncology: official journal of the American Society of Clinical Oncology 2015; 33(6): 594-601). The 6.2 months mPFS for balixafortide+eribulin in the Expanded Cohort of our trial is also higher than that reported for eribulin alone in any published trial recruiting similar patients (2.6-4.1 months). One year OS for the Expanded Cohort (75%) is encouraging with the corresponding result for eribulin alone being reported as 53.9%. The tolerability profile of balixafortide+eribulin allowed a long duration of treatment in this trial.

A further point of interest is that in 4 out of 9 patients who had PR in the Expanded Cohort, the time to onset of response was 1195164 days (3.965.4 months). Effective immunotherapies have been associated with later onset of objective response and, in some cases, pseudo-progression in the early stages of treatment because recruitment of T-cells to tumours causes a temporary increase in tumour size (Seymour L, Bogaerts J, Perrone A, et al. iRECIST: guidelines for response criteria for use in trials testing immunotherapeut. Lancet Oncology 2017; 18(3): e143-e52). Emerging data indicate that CXCR4 expression on immune cells may play a role in the distribution of tumour-infiltrating lymphocytes, and inhibition of CXCR4/SDF-1 signalling may favour the cytotoxic T cells function in tumours.

The patients were heavily treated for MBC previously and the promising safety, tolerability and anti-tumour activity observed in this trial can provide important insights into the direction of future research in the management of breast cancer. While the number of patients in the trial was limited, the sample size for the Expanded Cohort was similar to other translational studies and provides a reasonable precision of the anti-tumour effect.

Assessment of CXCR4 expression has not been reported consistently in the published literature, and different methods of detection are often used. No clear and standardised threshold for CXCR4 intensity of staining was reported at the time that the study was set up and, therefore, any level of positive expression for CXCR4 was accepted as an eligibility criterion to the study. All patients on the trial were CXCR4 positive, although most patients had weak CXCR4 expression. Responses were observed regardless of CXCR4 intensity of staining; however, this could be related to the limitation of the methodology and the fact that a large majority of the samples that were analysed were taken more than 1 year prior to enrollment (40/56).

1. A pharmaceutical combination comprising:
(a) a compound of formula I 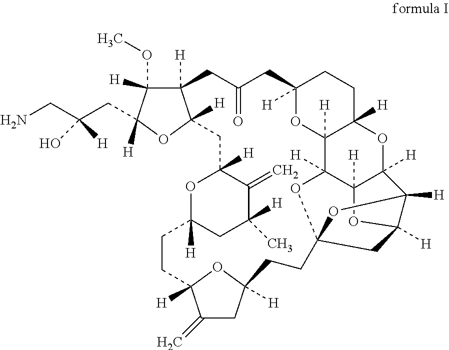
which has the chemical name 2-(3-amino-2-hydroxypropyl)hexacosahydro-3-methoxy-26-methyl-20,27-bis(methylene)11,15-18,21-24,28-triepoxy-7,9-ethano-12,15-methano-9H,15H-furo(3,2-i)furo(2′,3′-5,6)pyrano(4,3-b)(1,4)dioxacyclopentacosin-5-(4H)-one or a pharmaceutically acceptable salt thereof;
(b) cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof; and optionally
(c) one or more pharmaceutically acceptable diluents, excipients or carriers.

2. A pharmaceutical combination according to claim 1, wherein cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, is the acetate salt of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11.

3. A pharmaceutical combination according to claim 1, wherein said compound of formula I is the compound of formula Ia 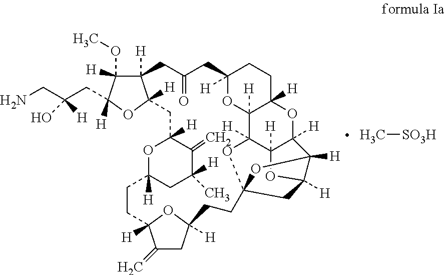
which has the chemical name 2-(3-amino-2-hydroxypropyl)hexacosahydro-3-methoxy-26-methyl-s(methylene)11,15-18,21-24,28-triepoxy-7,9-ethano-12,15-methano-9H,15H-furo(3,2-i)furo(2′,3′-5,6)pyrano(4,3-b)(1,4)dioxacyclopentacosin-5-(4H)-one methanesulfonate.

4. (canceled)

5. A method for the prevention, delay of progression or treatment of cancer in a subject in need thereof, comprising administering the pharmaceutical combination of claim 1 to said subject.

6. The method according to claim 5, wherein the cancer is selected from the group consisting of breast cancer, metastatic breast cancer, and relapsed metastatic breast cancer.

7. The method according to claim 5, wherein the cancer is HER2-negative relapsed metastatic breast cancer.

8. The method according to claim 5, wherein each of the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, is independently administered to the subject, wherein the compound of formula I or a pharmaceutically acceptable salt thereof is administered on days 2 and 9 and wherein cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is administered on days 1, 2 and 3, and on days 8, 9 and 10 of a 21-day cycle of administration.

9. The method according to claim 5, wherein the administration to the subject of the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, starts with a 21-day cycle which is repeated at least two times.

10. The method according to claim 8, wherein on day 2 and 9 of the 21-day cycle of administration the compound of formula I or a pharmaceutically acceptable salt thereof is administered about 15 to about 240 minutes after the end of the administration of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 for a pharmaceutically acceptable salt thereof.

11. The method accordingly to claim 8, wherein on day 2 and 9 of the 21-day cycle of administration the compound of formula I or a pharmaceutically acceptable salt thereof is administered over about 2 to about 10 minutes to the subject about 15 to about 120 minutes after the end of the administration of cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof which is administered over about 1 to about 3 hours.

12. The method according to claim 5, wherein the compound of formula I or a pharmaceutically acceptable salt thereof and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof, are administered to the subject intravenously.

13. The method according to claim 5, wherein the compound of formula I or a pharmaceutically acceptable salt thereof is administered to the subject at a dose between about 0.1 and about 10 mg/m² and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 or a pharmaceutically acceptable salt thereof is administered at a dose between about 0.1 and about 10 mg/kg.

14. The method according to claim 5, wherein the compound of formula Ia is administered to the subject at a dose between about 1 and about 2 mg/m² and cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11 is administered to the subject at a dose between about 4.5 and about 8 mg/kg.

15. The method according to claim 5, wherein the subject who has cancer is (i) refractory to at least one chemotherapy treatment, or (ii) is in relapse after treatment with chemotherapy, or a combination thereof.

16. A kit of parts comprising a first container, a second container and a package insert, wherein the first container comprises at least one dose of a medicament comprising a compound of formula I or a pharmaceutically acceptable salt thereof or a compound of formula Ia, the second container comprises at least one dose of a medicament comprising cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, or a pharmaceutically acceptable salt thereof, and the package insert comprises instructions for treating a subject for cancer using the medicaments, wherein the cancer is selected from the group consisting of breast cancer, metastatic breast cancer, and relapsed metastatic breast cancer.

17. A pharmaceutical combination according to claim 2, wherein said compound of formula I is the compound of formula Ia 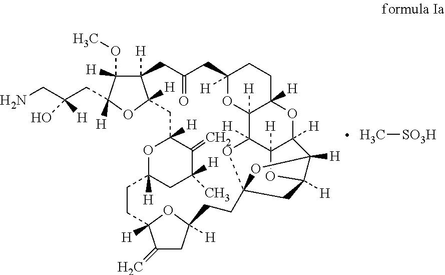
which has the chemical name 2-(3-amino-2-hydroxypropyl)hexacosahydro-3-methoxy-26-methyl-20,27-bis(methylene)11,15-18,21-24,28-triepoxy-7,9-ethano-12,15-methano-9H,15H-furo(3,2-i)furo(2′,3′-5,6)pyrano(4,3-b)(1,4)dioxacyclopentacosin-5-(4H)-one methanesulfonate.

18. A method for the prevention, delay of progression or treatment of cancer in a subject in need thereof, comprising administering the pharmaceutical combination of claim 2 to said subject.

19. A method for the prevention, delay of progression or treatment of cancer in a subject in need thereof, comprising administering the pharmaceutical combination of claim 3 to said subject.
